(19) United States
(12) Patent Application Publication
(10) Pub. No.: US 2009/0197322 A1
(43) Pub. Date: Aug. 6, 2009

(54) SOLAR PLANT EMPLOYING CULTIVATION OF ORGANISMS

(76) Inventor: Arnold J. Goldman, Jerusalem (IL)

Correspondence Address: MILES & STOCKBRIDGE PC, 1751 PINNACLE DRIVE, SUITE 500, MCLEAN, VA, 22102-3833, US

(21) Appl. No.: 12/246,422

(22) Filed: Oct. 6, 2008

Related U.S. Application Data
(63) Continuation of application No. PCT/US07/66195, filed on Apr. 6, 2007.
(60) Provisional application No. 60/744,358, filed on Apr. 6, 2006.

(51) Int. Cl. C12N 1/12; C12M 1/00; F01D 15/10; F03G 6/06

(52) U.S. Cl. 435/257.1; 435/292.1; 290/52; 60/641.15

(57) ABSTRACT

A method of growing algae is described that is part of a cogenerational energy production plant in which solar energy from a solar collecting and concentrating field is used to provide photonic energy for the growth and stress phase of algae as well as to provide heat for driving a turbine. The supplementary energy for the power plant is provided by natural gas and by biomethane that is produced by fermentation of the algal biomass. The carbon dioxide that is a by-product of the combustion of both the natural gas and the biomethane is recycled to provide the carbon source for the algal growth.

### FIELD OF THE INVENTION

The present invention relates to the cogeneration of products such as electricity, fuel, and extractants as well as related processes, articles of manufacture, devices, and systems that are usable for cogeneration.

### BACKGROUND

Cogeneration is the generation of useful products in a combined process or related processes. For example, some types of cogenerating electrical plants use heat engines which produce low temperature energy as a byproduct of the subprocess of generating electricity and make that low temperature energy available for useful purposes such as residential heating. Cogenerating electricity using incident solar light and simultaneously growing a boiler feedstock, such as an algae, within a fluid medium which flow through solar collectors is known. The solar energy is used to heat water to a first temperature, and the algae is combusted to raise the temperature of the water to a greater temperature sufficient for boiling the water for the creation of steam which is then passed through a steam turbine coupled to a generator for the generation of electricity therewith. Simultaneously, or sequentially, fossil fuel, such as natural gas, may be used to power a gas turbine coupled to an electrical generator to also provide electricity. Additionally, it is known to extract materials from the algae, such as oils therein, which may be used to fuel the gas turbine.

One problem frequently encountered in solar energy generation is the efficiency of the system, i.e., the cost of electricity generated therewith which is a direct function of the underlying cost of the solar generating facility. The cost of the equipment needed for solar electrical generation in combination with the market price for the electricity generated is substantially higher than an equivalent generation capability using fossil fuel. As a result, solar energy has not been really accepted except in situations where fossil fuel based generation is not feasible or where government mandates or subsidies dictate solar generation.

### SUMMARY OF THE INVENTION

The present invention provides methods and apparatus for the generation of electrical power using solar energy, as well as the manufacture through biological processes of materials useful for non-electricity generating applications.

In one aspect, a solar electrical power generating facility is provided, which includes a solar field, an electrical generation facility, and an extraction facility. The solar field includes a fluid medium, into which solar insulation may be provided, and within which is grown a medium, such as an algae, which produces, during growth, including cellular multiplication, at least one material which may have a utility other than for the production of electricity.

In a method, the medium is subjected to solar insulation and thus grown to produce additional medium and as a byproduct a material having a utility other than for the production of electricity. In a further aspect, the material having a utility other than for the production of electricity is extracted from the medium, and the portion of the medium remaining after extraction may be used as a fuel for the generation of heat for use in the generation of electricity using either a steam turbine, a gas turbine or other generation facility.

In an additional aspect, an article of manufacture includes a material created as a consequence of the growth of a medium used in a solar field, including where the medium is further useful, after the extraction of the article of manufacture therefrom, as a fuel for the generation of electricity.

According to an embodiment, a method of generating electricity includes: concentrating sunlight to a first level of solar flux and selecting a portion of the spectrum of the light resulting therefrom and using it to photostress an organism. In another embodiment, the selecting includes reflecting concentrated light from a hot mirror. In yet another embodiment, the concentrating includes reflecting light from a heliostat array. In yet another embodiment, the method also includes receiving the portion of the spectrum on angled surfaces forming a cascade over which liquid media, containing growth culture, trickles.

According to another embodiment, a method of generating electricity, includes: directing sunlight onto a series of angled surfaces forming a cascade and directing liquid media carrying living organisms repeatedly over the surfaces to photo stress the organisms. Preferably, the directing includes concentrating the sunlight to a first level of solar flux. Preferably also, the concentrating includes reflecting light from a heliostat array. In an alternate embodiment, the directing includes selecting a portion of the solar spectrum. In yet another embodiment, the selecting includes reflecting concentrated light from a hot mirror.

According to another embodiment, a bioreactor has at least one member having an array of angled surfaces forming a cascade. The angled surfaces are irregular such that fluid flowing thereover is rendered turbulent. A recycling channel directs fluid from the bottom of the cascade to the top such that the fluid flow down the cascade repeatedly. Preferably, the at least one member forms gutters at intermediate points which are effective to spread fluid across the angled surfaces.

According to another embodiment, a method of generating electricity, includes: concentrating a first portion of sunlight onto a receiver which conveys a working fluid to an electrical generator and conveying a second portion of the sunlight onto a bioreactor, the bioreactor being configured to grow an organism in the dark by selectively blocking sunlight predefined times or in response to detected conditions and selectively unblocking the sunlight at other times or under other detected conditions.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 shows a basic layout of a plant that produces electricity and employs the cultivation of biological cells for form a cogeneration facility.

FIGS. 2A through 2E show various process variations for generation of useful products and based respectively on mixotrophic or autotrophic cultivation.

FIG. 3 shows the major steps involved in the downstream processing of the various useful products.

FIGS. 4 to 9 show downstream processing for extraction of useful products consistent with respective products of cultivation.

FIG. 10 shows a combination reactor/solar receiver for cultivating and photo-stressing the products of cultivation.

FIG. 11 shows a detail of a preferred embodiment of the reactor/solar receiver of FIG. 10.

FIG. 12 shows a cogeneration plant which produces various products including thermal and electrical energy,

### DESCRIPTION OF THE EMBODIMENTS

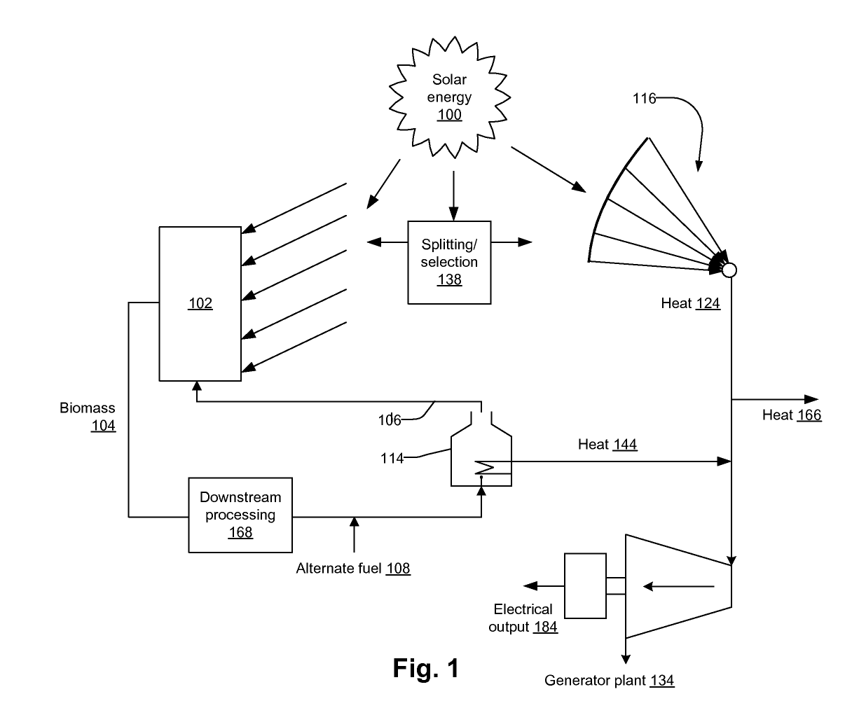

FIG. 1 shows a basic layout of a plant that produces electricity and employs the cultivation of biological cells for form a cogeneration facility. Solar flux from the sun 100 is gathered by a high temperature thermal collector component 116 such as a concentrating collector system and converted to thermal energy 124 which may be conveyed by a suitable heat transfer medium. The thermal energy 124 is used to drive a generator plant 134 to produce electricity 184. The generator pant 134 can also receive heat by burning biomass 104 from a bioreactor 102 or an alternate fuel 108 such as natural gas. The bioreactor receives solar flux from the sun 100 at a suitable concentration depending on its design. Carbon dioxide from the combustion products from a boiler or other heat generator 114 can be supplied to the bioreactor 102 to be consumed in the production of biomass. The bioreactor 102 is preferably capable of producing other useful products such as nutrients.

The bioreactor 102 may provide for various stages in the production of a biological material or by-product. For example, in an embodiment, the biological material is astaxanthin which is produced by cultivation and harvesting from Haematococcus. A suitable process begins with the provision of fresh culture from a culture stock such as from a lab facility which is mixed with nutrients and a media. The culture, media, and nutrients are fed into a suitable reaction vessel at a feed or supply rate determined by the vitality of the culture in the reaction vessel and the chemical balance of the nutrients. In the embodiment employing Haematococcus cells, these are maintained in their vegetative state in the dark so that they grow heterotrophically. The culture is bled and filtered, preferably also automatically analyzed, cleaned, and the media rebalanced and returned to the dark bioreactor. This may be done as a batch process on a periodic schedule or as part of a continuous process. Thermal energy 166 may be used as a product for other uses as well such as sterilization of the bioreactor 102 and for a thermally-activated cooler, which has uses in the cultivation and harvesting of biological products as discussed below.

Media, nutrients, and cells are drawn from the dark reaction vessel and fed to a stressing reaction vessel either continuously, on a quasi continuous basis depending on conditions such as the availability of sunlight for photostressing, and/or in batches. In the Haematococcus embodiment the cells are stressed by exposing them to sunlight in a stressing reactor, causing the cells to produce astaxanthin. Preferably, also, a continuous stream of media, cells and nutrients is tapped from the stressing reactor and passed through a milking chamber together with a lipophilic solvent where the solvent and the biomass are mixed. The solvent is selected based on the cell type and the extractant. The solvent containing the extractant (extracted molecules) passes from the milking chamber 122 to a downstream processing system discussed below. After extraction the cells are returned to the stressing reactor and the solvent recycled until saturation.

The biomass 104 derived from the bioreactor 102 is processed in the downstream processing 168 which provides such processes as concentration of biomass. cell rupturing, extraction of algal contents, centrifugation of biomass, spray drying of whole algal cells, formulation of cell contents, conversion of biomass and bio-organic residues into combustible bio fuels, milking of biomass to produce useful extractants. The non-fuel output 129 from the downstream processing facility 127 includes but is not limited to high value chemicals such as astaxanthin, J3-carotene, coenzyme.

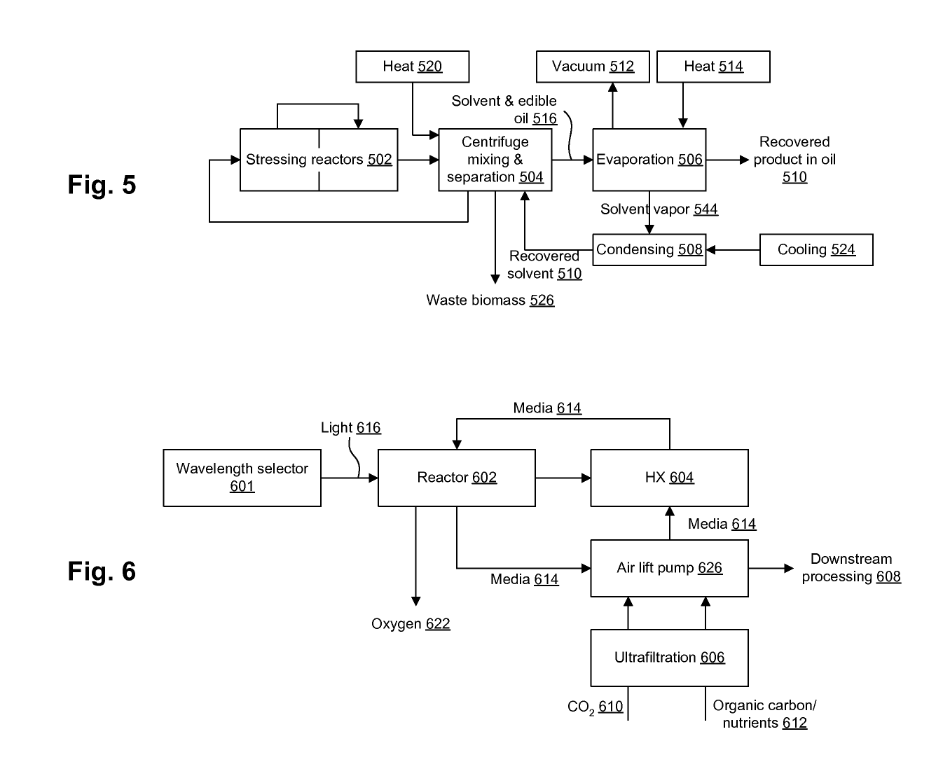

In an alternative system, the photo-stressing reactor is divided into two halves: a first reactor in which the cells are stressed to produce astaxanthin and milked in the manner described above; and a second reactor which receives the cells after the milking processes. In this way the production of astaxanthin and the milking of the cells can be optimized. See FIG. 4.

From this stressing/milking stage the culture is continuously fed into a secondary stress reactor where the cells undergo a final stress sequence and are then passed on as whole cells for downstream processing.

Three routes are available in the down-steam processing of the medium, depending on the form of the end-product: whole cells, extractant from whole cells formulated into oil, extractant in oil from milking process. In the case where the whole cells are harvested (as shown in FIG. 5) the first step is to dewater the culture by continuous centrifuging. In the case where cell disruption is necessary, to either facilitate higher bioavailability from the whole cells or to expose the lipophilic target to the extraction solvents, the wet biomass is passed through a mechanical high pressure hydraulic disrupter which shatters the cell walls. The fragmented cells are then either passed onto pasteurization and spray drying or to the extraction unit. An efficient environmentally friendly method has been developed for extracting out the lipophilic products from wet biomass thereby circumventing the expensive need to dry (U.S. Pat. No. 6,818,239). The result of this extraction process yields products that are directly formulated into edible oils ready for marketing.

The alternative process involves product that has been extracted out from live cells using the milking technique. If the extracting solvent is a non-edible solvent then the dissolved lipophilic product is mixed with a suitable edible oil and then passed through an evaporation tower that removes the lower boiling extracting solvent leaving the desired product dissolved in oil. See FIG. 4.

The Products

The cost of the products produced by solar-reactor field depends on the following factors:

1. Biological
Rate of growth (productivity) of the organism;
Culture densities;
Concentration of desired product per cell;
Ease of extraction; and
Bioavailability.

2. Physical
Use of reactors;
Use of the solar spectrum;
Light path lengths;
Light/dark transition in—mixing; and
Light intensity/culture density ratio.

The following technologies will be brought into play in order to maximize each of these factors and thus the overall productivity:
1. Mixotrophic cultures;
2. Selective wavelengths of collected sunlight;
3. Continuous milking of lipophilic products;
4. Strain improvement to increase yields and, in the case of Haematococcus, to increase extractability of astaxanthin;
5. High flux constant daytime irradiation;
6. Maximal exposure of cells to light by cascading thin film flows; and
7. Ultra-high cultural concentrations.

### Astaxanthin

The embodiment includes the production of astaxanthin from the micro-algae Haematococcus. This organism has proven to be capable of metabolizing astaxanthin in higher concentrations (6% pigment content) than any other species. (For instance, an alternative natural source of this carotenoid is the yeast Phaffia rhodozyma which produces 0.4% pigment content.).

The developmental cycle of Haematococcus occurs in two distinct stages: macrozooids and haematocysts. The former predominate in liquid cultures when there are sufficient nutrients, but when environmental conditions become unfavorable (intense light, salt, large temperature fluctuations) they begin to develop heavy resistant cell walls and accumulate astaxanthin. When conditions become favorable again the cysts give rise to motile microzooids that grow into the macrozooid stage. It has been found that the specific rate of astaxanthin accumulation is a function of the photon flux density that the Haematococcus cultures are exposed (Lee, Y. K. et al, “Accumulation of Astaxanthin in Haematococcus Lacustris,” J. Phycol. 27: 575 (1991)).

Recent studies (Zhang, X. W. et al., “Kinetic models for astaxanthin production by high cell density mixotrophic culture of the microalga Haematococcus pluvualis,” J. Ind. Microtech. Biotech. 23: 691 (1999); Hata, H. et al., “Production of astaxanthin by Haematococcus pluvialis in a sequential heterotrophic-photoautotrophic culture,” Journal of Applied Phycology. 13: 395-402 (2001); Barbera, E. et al., “Modelling mixotrophic growth of microalgae Haematococcus lacustris,” Afinidad. 59: 386-390 (2002); and Kobayashi, M., Kakizono, 1., Yamaguchi, K., Nishlo, N. and Nagai, S., J. Fermen. Bioeng. 74: 17-20 (1992)) have shown that Haematococcus can grow heterotrophically in the dark, utilizing organic carbon and oxygen as primary sources of nutrition, mixotrophically during the night, utilizing organic carbon, oxygen for heterotrophic metabolism, and light and carbon dioxide for photoautotrophic metabolism, simultaneously. Furthermore it was found that the specific growth rate of the mixotrophic condition (organic carbon+light) corresponded well to the sum of the specific growth rates of the heterotrophic (organic carbon+dark) and autotrophic (no organic carbon+light) conditions. Growing Haematococcus under these conditions will allow full and continuous use of the dedicated bioreactor field to maximize production of astaxanthin.

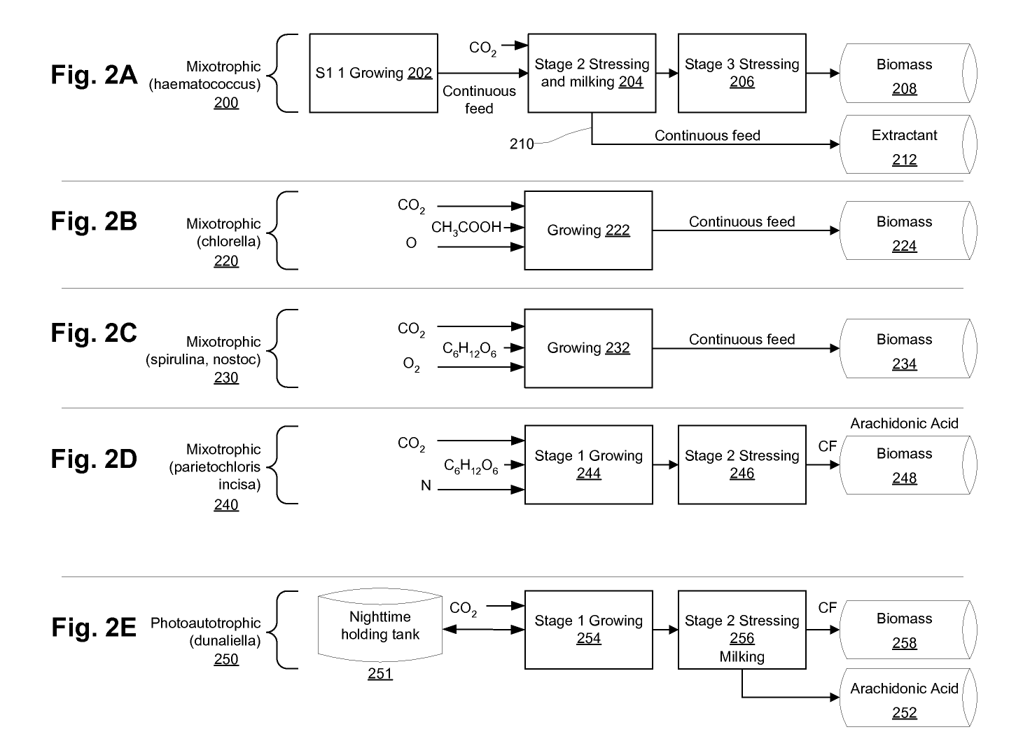

In FIG. 2A, a process 200 of biomass and extractant production begins with the a growth stage of Haematococcus which is confined to a dark bioreactor 202 where growth takes place by heterotrophic cultivation. The advantage of growing the Haematococcus cells in the dark is that it is possible to delay the onset of the encystment stage thus allowing for much higher vegetative cell densities. Fresh nutrients are continually added from nutrient storage tanks to preserve the vitality of the medium. Old medium is siphoned off through a cross-flow filtration unit, automatically analyzed, recharged and stored in a recycled nutrient storage tank.

From this dark reactor the cells are continuously fed into a Multiple Cascade Photo-Stressing Reactor (MCPSR) 202 for photo-stressing. In order to increase the density of the culture in the photo-stressing phase, a cross-flow filtration unit also acts to filter off excess medium as the cells pass from one reactor to the next. The density of the culture ceases to grow at this stage although low amounts of carbon dioxide will be necessary to allow for full mature development of the haematocysts. Stressing will occur during the day by photo-stressing and at night by nitrogen stressing (Choi, Y. E., et al., “Evaluation of factors promoting astaxanthin production by a unicellular green alga, Haematococcus pluvialis with fractional factorial design,” BioTech Proci. 18: 1170-1175 (2002)).

It has been shown that the rate of growth of algae and the rate of carotenoid production are sensitive to different wavelengths of light (Ashkenazi, R., “The response of Dunaliella bardawil to the natural changes in the sunlight spectrum and intensity,” PhD dissertation, The Weizmann Institute of Science (1999); Tekoah, Y., “The effects of the spectrum and concentration of light on the productivity of microalgae,” PhD dissertation, The Weizmann Institute of Science (1994); Kobayashi, M. et al., “Effects of light intensity, light quality, and illumination cycle on astaxanthin formation in a green alga, Haematococcus pluvialis,” J. Ferm. Bioeng 74: 61-63 (1992); and Park, E. K. et al., “Astaxanthin production by Haematococcus pluvialis under various light intensities and wavelengths,” J. Microbiol. BioTech 11: 1024-1030 (2001)). It was found that whereas red light is critical for the growth phase, blue light promotes stressing and carotenoid development. Therefore the preferential incident light in a MCPSR will be predominantly blue for maximal production of astaxanthin. The use of the MCPSR allows for very short light path lengths, maximal use of the incident light, minimal photo-inhibition, and ultra-high culture densities.

The preferred mode of harvesting the astaxanthin is by continuous in situ extraction using the method developed by Hejazi and Wijffels for Dunaliella (Hejazi, M. A. et al., “Milking microalga Duneliella salina for β-Carotene production in two-phase bioreactors,” BioTech. BioEng 85: 475-481 (2004) and Leon, R. et al., “Microalgae mediated photoproduction of β-carotene in aqueous-organic two phase systems,” BioMol. Eng 20: 177-182 (2003)). This method is comparable to milking: the astaxanthin is removed by means of a co-solvent that selectively dissolves and removes the lipophilic carotenoid from within the cellular lipid sacs. The method allowed cells to be milked for more than fifty days with continuous production of new extractant 212, in this case, astaxanthin within the same viable cells. The advantages of such a method are:

High volumetric productivity

Elimination of cell harvesting and concentration

Elimination of cell destruction

Simplification of purification.

According an embodiment, the astaxanthin is milked external to the MCPSR 204. The solvent of choice will be either dodecane or edible oil for the example embodiment. The process for recovering the astaxanthin is described below. To take advantage of the milking technique, a strain of Haematococcus may be used that has either a thin cyst wall or preferably no cyst wall and in effect is similar to a wall-less Dunaliella algal cell.

As stated, the extraction of the astaxanthin takes place immediately adjacent to the reactor in a mixing and separation chamber. The organic phase is recirculated until reaching saturation and then transferred to the downstream processing unit. The astaxanthin-depleted culture is recycled back into the stressing chamber.

To ensure the vitality of the culture, a continuous volume is bled off into a secondary MCPSR 206. The fully stressed cells from this reactor are bled off directly to the downstream processing unit to produce biomass 208 which may be fluid separated and subjected to whole-cell drying.

### Chlorella

Chlorella biomass may also be cultivated from the micro-algae Chlorella vulgaris in a process 220 shown in FIG. 2B. A useful substance for human health, and therefore a desirable product, in the Chlorella cell is β-1,3-glucan, which is an active immunostimulator and has many other functions, such as free radical scavenger and a reducer of blood lipids. The Chlorella grows mixatrophically in a reactor 222 (Ogawa, T. et al., “Bioenergetic analysis of mixotrophic growth in Chlorella vulgaris,” Biotechnol. Acta. 23: 1121 (1981)) using oxygen and acetic acid as the organic carbon source (carbon dioxide is supplied through oxidative decomposition of acetic acid by the Chlorella cells) in which the photosynthetic and the heterotrophic growth mechanisms function independently.

The Multi-Cascade Bio-Reactor (MCBR) 222 is shown for Chlorella. The reactor 222 is continuously supplied with fresh medium while high density culture 224 is bled off and passed on to the downstream processing unit. In a downstream process, in which cells are centrifuged, the cells are subjected to high pressure cell disruption methods to break open the multi-layered cellulose cell wall in order to improve the bio-availability of its contents. After that the cells are spray dried and packaged. Heating the cells to 110° C. is preferably performed to denature the active chlorophylase and prevent the build up of harmful phaeophorbide.

### Spirulina.

A process 230 in FIG. 2C is suitable for obtaining useful products from species such as a species of the cynabacteria Spirulina platensis. This is grown in part of bio-reactor 232 in a similar manner as described for Chlorella. Spirulina can also be grown mixotrophically during the day and heterotrophically by night (Marquez, F. J. et al., “Growth characteristics of Spirulina platensis in mixotrophic and heterotrophic conditions,” J. Ferm. BioEngin 76: 408-410 (1993)) in which the sum of the cell concentration of the mixotrophic cultures corresponds well to the sum of the autotrophic and heterotrophic cell concentrations. In that case, the day time cultures are fed on glucose with oxygen supplied via the air in the airlift pump for the heterotrophic growth cycle and carbon dioxide supplied for the autotrophic growth cycle. The biomass 234 is bled off continuously and transferred to a downstream processing plant 168 for drying and packaging. In an embodiment of a process for Spirulina there is no cell disruption. Another form that may be used is the strain Spirulina flos-aquae, which is the form found naturally in Klamath Lake, Oreg. and which yields 2000 tons of algae a year. Growth of this species in the bio-reactors yields a product free from contamination by neurotoxins from competing strains.

### Nostoc

The process 230 is suitable for Nostoc commune which is processed according to another embodiment. Nostoc commune is an edible blue-green alga forming the spherical macrocolony, which has been consumed as a potent herbal medicine and health food in Asia for centuries. Very recent medical research have revealed that Nostoc commune contains a number of bioactive compounds that can kill cancer cells and HIV, as well as controlling hypertension, depressing LDL-cholesterol level, and helping exhaust relief. Due to growing awareness on its nutraceutical and pharmacological value, the Nostoc commune has received increasing attention, and the market demand has grown drastically during the last decade from $10 million to $150 million annually. However, further expansion of the Nostoc commune market is limited by the primitive production method, which is to harvest Nostoc commune from its natural habitat. In this way, the production of Nostoc commune is heavily dependent on the climate conditions and the quality of Nostoc commune varies greatly. Because of these issues, there is an urgent need for more reliable production method that can produce high quality Nostoc commune to meet the increasing market demand (Fan, L., private communication).

Parietochioris incisa

In a further embodiment, Arachidonic acid (AA) is derived 240. AA is an essential fatty acid in human nutrition and a precursor for the biologically active prostaglandins and leukotrienes which have important functions in the circulatory and central nervous systems. AA is found as a component of human milk and has therefore become a valuable additive to formulated, artificial baby food. A recently discovered algal source of AA has been reported by Richmond et al. (Cheng-Wu, Z. et al., “Characterization of growth and arachidonic acid production of Parietochloris incisa comb. nov (Trebouxiophyceae, Chlorophyta),” Journal of Applied Phycology. 14: 453-460 (2002)) with area yields of 1 gm per m² per day.

According to the embodiment a mixotrophic strain of Parietochioris allows for 24 hour growth cycles. The first MCBR 244 is for photoautotrophic and heterotrophic growth with a daylight supply of carbon dioxide an optimized supply of organic carbon (glucose or acetate) and nitrogen (nitrate salts). At night the carbon dioxide feed stream is closed. A steady bleed-off from the growth bio-reactor 244 introduces fully grown cells into the stress bio-reactor 246 which irradiates the culture with blue light while inducing nitrogen starvation. These three stressing factors: high intensity light, blue light, and nitrogen depletion are preferred because they provide high AA yields. From the stressing bio-reactor 246 a continuous bleed-off passes the AA-rich culture to the down stream processing unit for medium concentration (by centrifugation CF, for example) and dry spraying of the biomass 248.

### Beta-Carotene

In another process embodiment 250, Dunaliella bardawil is processed. Dunaliella bardawil is a halotolerant species that, under stressful environmental conditions, can produce more than 12% β-carotene per dry weight biomass. Referring to Fig, an arrangement for the continuous growing and stressing of Dunaliella employs a nighttime holding tank 251. In the preferred embodiment, Dunaliella undergo growth in an MCPBR with a continuous bleed into a stressing photo-reactor 256. Stressing will be by predominately blue light.

In a similar manner described above for Haematococcus, the β-carotene will be harvested by the continuous milking process in a mixing and separating reactor adjacent to the stressing chamber 256. During night times the contents of the two reactors are transferred to holding tanks 251. For the bulk culture that is in growth mode, nutrient conditions are such that the organism is maintained in a healthy state. The bulk culture that is in the stressing mode will continue to be stressed through nitrogen deprivation. At day break the volumes in the holding tanks 251 are transferred back into their respective bioreactors 254, 256. During the night hours the empty MCPBRs are used for heterotrophic growing of other species.

### Downstream Processing

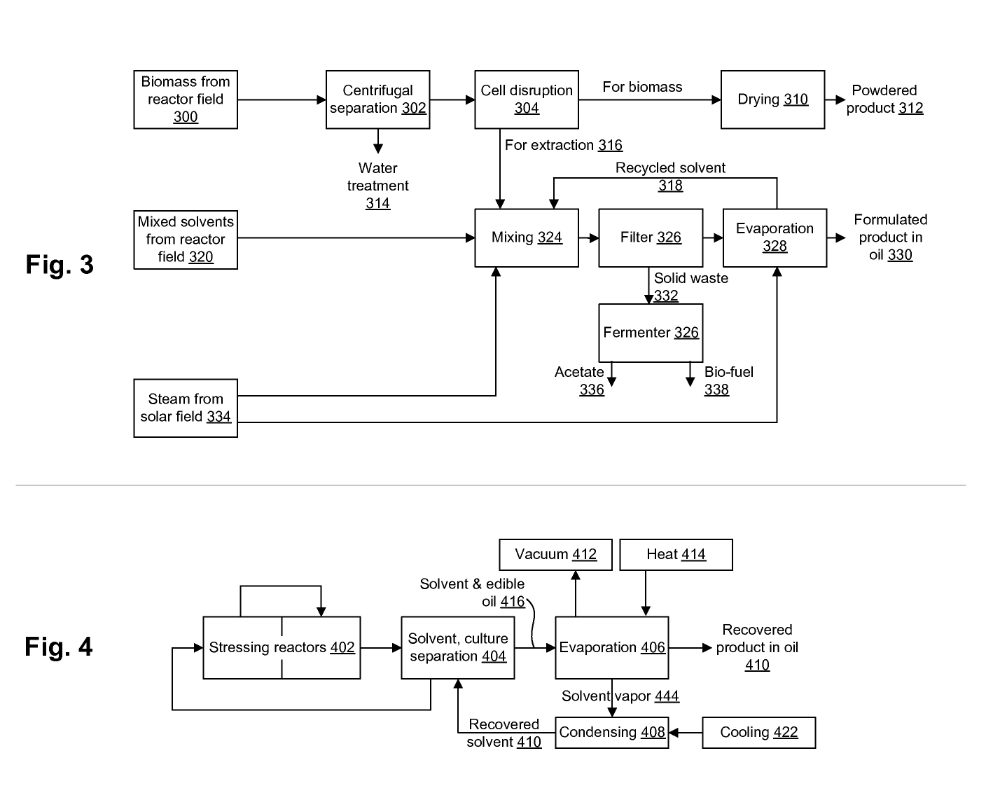

FIG. 3 shows the major steps involved in the downstream processing of the various products 300. Particular examples using steps of the processing elements of FIG. 3 are shown in the later figures. Dewatering, that is concentrating the biomass, is performed by continuous-feed centrifuging 302. Mechanical cell disruption 304 is preferably performed step in the case of a regular Haematococcus species that forms a tough cyst cell wall. In an embodiment, disruption is performed by high pressure homogenizers. In an embodiment, dry biomass is the end product 312, in which case, disrupted cell mass undergoes a washing and pasteurization process, and then passes to the spray drier for final drying and packaging.

In the same or alternate embodiment, astaxanthin is a product in which case the disrupted cells are passed to an extraction processor, preferably using the co-solvent method described in U.S. Pat. No. 6,818,239, hereby fully incorporated by reference, for extracting the astaxanthin into edible oil without the use of environmentally undesirable solvents. The volatile co-solvent is evaporated 328 off in a low-pressure evaporating tower leaving astaxanthin formulated into an edible oil. The co-solvent is fully recovered and recycled.

Disrupted culture may be missed with solvents 324, filtered 326, and sent to an evaporator tower 326. Solid waste 332 may be fermented 326 to generate acetate 336 and/or bio-fuel 338.

In FIG. 4, a milking process in which culture is mixed lipophilic solvent 404, which, carotenoid-rich, is mixed 324 with edible oil 416, and transferred to the evaporation tower 406 where the lipophilic milking solvent is evaporated off leaving recovered product in edible oil 410. The evaporation process preferably is done under vacuum 412 and/or with supplemental heat 414, both of which may be generated using thermal or electrical energy cogenerated by the facility of FIG. 1. The solvent is recovered 408 and recycled 410 and product recovered 410. Solvent recovery from solvent vapor 444, in an embodiment, employs a condenser 408, which, in an embodiment, is provided using cooling 524 cogenerated heat or electricity from the facility of FIG. 1.

In FIG. 5, a centrifuge separation process is used in which culture centrifuged 504 and mixed with and solvent and edible oil 516, and transferred to the evaporation tower 506 where the lipophilic milking solvent is evaporated off recovered product in edible oil 510. The evaporation process preferably is done under vacuum 512 and/or with supplemental heat 514, both of which may be generated using thermal or electrical energy cogenerated by the facility of FIG. 1. The solvent is recovered 508 and recycled 510 and product recovered 510. Solvent recovery from solvent vapor 544, in an embodiment, employs a condenser 508, which, in an embodiment, is provided using cooling 524 cogenerated heat or electricity from the facility of FIG. 1. Waste biomass from centrifugation 504 is recovered and is used, in an embodiment, to generate methane to fuel the generator plant in the cogeneration system of FIG. 1.

### The Bio-Reactors

A particular embodiment of Solar Bioreactor (SBR) is approximately 50 meters long, 5 meters wide, and 2 meters deep hermetically sealed unit. Three sides of the unit are stainless steel and the top surface is highly transparent. The SBR contains 90 algae production plates of approximately 20 m² each. Each bioreactor contains adjacent to it two 18 m³ storage tanks one which stores the working medium not used during the day shift and one that stores the working media not used during the night shift. The corrugated surfaces allow the liquid medium to flow down as a thin film over the protrusions and with micro-mixing in the indentations.

FIG. 6 shows such an SBR 602 with plates (not shown here, but see more details in the further embodiments disclosed below) used for the growing (green) stage in say Haematococcus. The incident light 616 is preferably selected using a suitable wavelength selector 601 to be primarily in the red region of the spectrum for increased growth rates. An air-lift pump 626 continuously circulates the culture from the bottom of cascade plates (described below) to the top of the SBR 602. Carbon dioxide is filtered and introduced during the daylight hours into the culture immediately upstream of the airlift pump 626.

Preferably organic carbon nutrients 612 are provided in the recirculating culture media 614. In a mixotrophic species organic carbon such as acetate or glucose is continuously fed into the flow. Injected materials are preferably ultrafiltered 726. At nighttime or when photosynthesis is not taking place the carbon dioxide input 610 is closed off. Excess heat is removed by a heat exchanger 604 on the recycling pathway as shown. The energy for the cooling system is preferably provided as a by-product of the heat produced by the solar portion of the cogeneration system of FIG. 1.

A proportion of the bioreactor volume is continuously bled off for processing 608 or to a photo-stressing chamber (not shown here).

FIG. 7 shows a MCBR (Multiple Cascade Bioreactor) for heterotrophic growth only. In this case insolation is not provided to the reactor chamber 702, so the wavelength selector 701 is shown in broken lines since it is not in the process loop. Also, only organic carbon 712 is introduced into the circulation flow. Media 714, air lift pump 706, heat exchanger 704, and ultrafiltration 726 etc. are similar to the respective counterparts of FIG. 6.

FIG. 8 shows a photo-stressing MCBR such as used for the production of astaxanthin in Haematococcus cells. A wavelength selector 801 provides that the intense irradiating light 816 is selected to be strong or as much as substantially exclusively in the ultra-violet to blue region of the solar spectrum, thus promoting greater yields of secondary pigments. Nitrogen 812 is added to stress the medium that also adds to increase productivity. Other components are similar to counterparts shown in FIG. 6.

FIG. 9 shows the same photo-stress bioreactor with the addition of a milking chamber 936 following the circulation pump 926. Continuous quantities of culture are shunted into a mixing chamber containing a non-aqueous solvent of choice (dodacane) where efficient mixing takes place. After an optimal period of time the two phases are separated, the aqueous culture phase is recycled back into the stressing chamber 902 and the non-aqueous layer is left in the chamber until saturated. It is then passed on to the down-stream processing unit. Other components are similar to counterparts shown in FIG. 6.

Each bioreactor preferably has two holding tanks (not shown) adjacent to it for either holding the solutions at night, or for cleaning of the chambers during downtime.

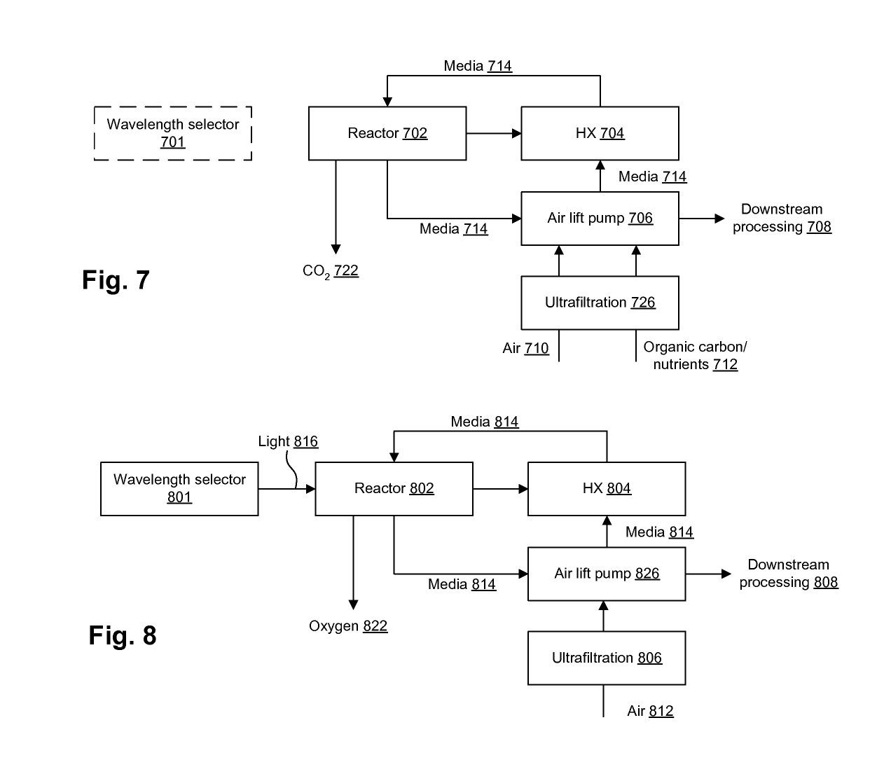

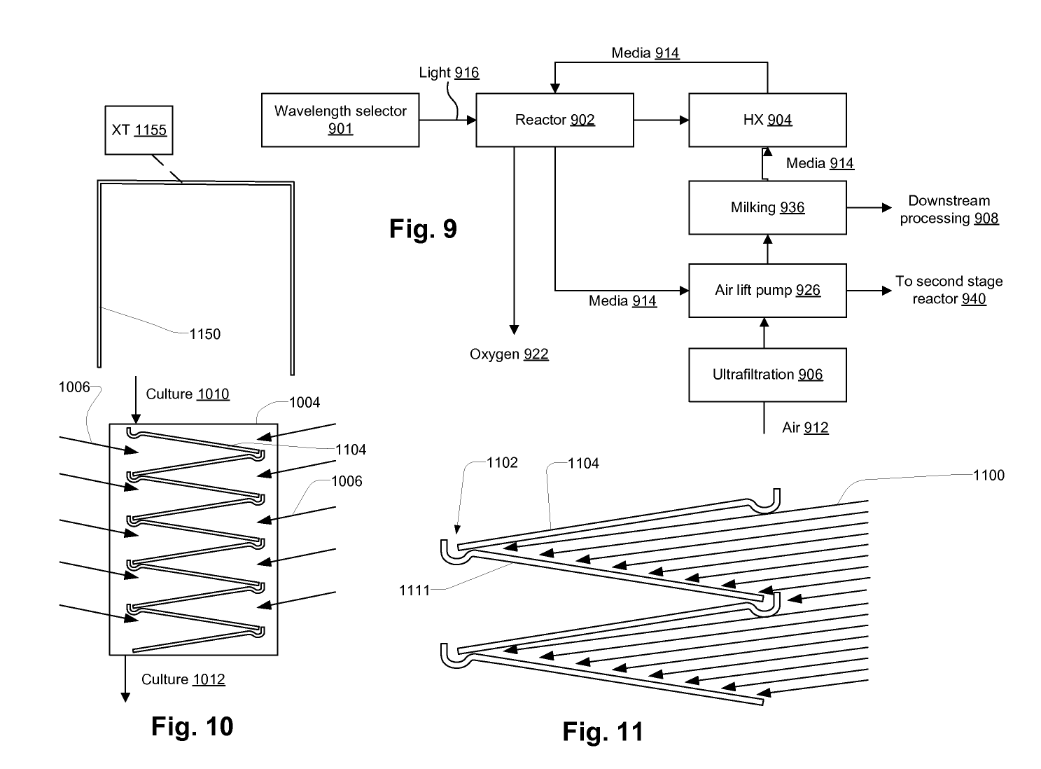

FIGS. 10 and 11 show an embodiment of the Closed Loop BioReactor 1003 in which the cascading plates 1018 are stacked one upon the other in an accordion-type arrangement where the culture 1010 flows down on the top-side of one plate into a gutter 1102 which overflows on to the top surface of the plate 1111 below. Each plate 1104 is preferably made of a non-flat surface that causes turbulent flow causing the algae cells alternative exposure to light and dark cycles. Upon reaching the end of the descent the exiting culture 1012 is transported to the top by means of a pumping system (not shown here). Sunlight 1100 is directed an angle relative to the plates 1104 and may therefore be concentrated relative to normally incident sunlight for optimal treatment, due to cosine loss. The concentration may be provided by conveying light using a concentrating facility.

This 1004 and the various bioreactors described herein may be provided with movable solar shield or cover 1050 that block sunlight to permit the growing phase in the absence of sunlight. In a preferred embodiment of the bioreactors, the same bioreactor, such as that shown in FIG. 10, which exposes the culture to sunlight can be used to condition the culture and media for nighttime growth. Such a cover 1150 may be activated automatically by a suitable controller 1155, for example in response to detected sunlight or a daily schedule.

### The Cogeneration Energy Power Plant

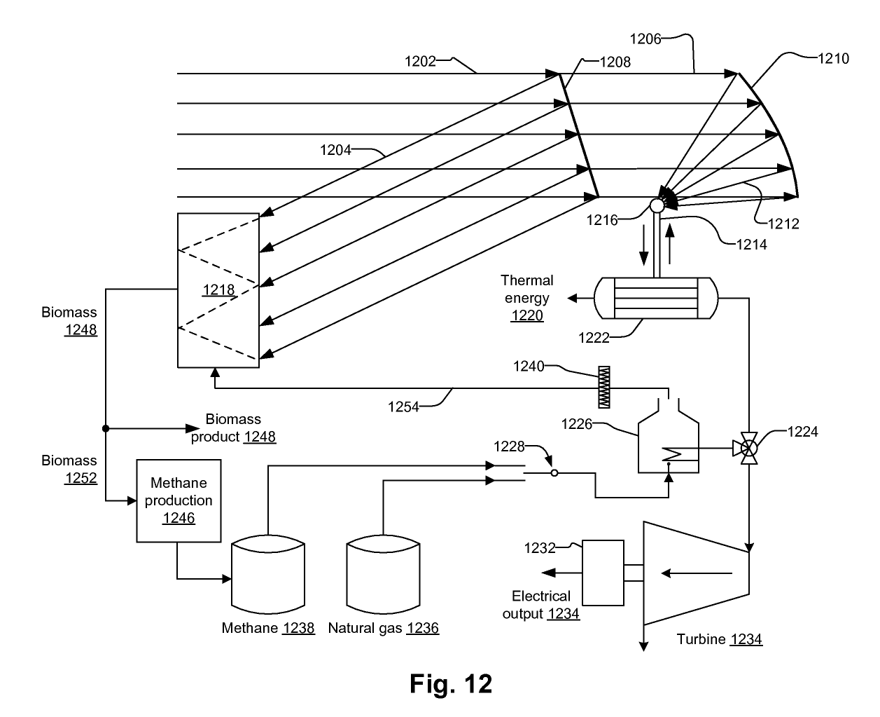

FIG. 12 shows the split use of, preferably concentrated, solar flux 1202. The latter is, in an embodiment, provided by a concentrating array of heliostats which transmit light to a wavelength selecting filter 1208. Light of wavelengths that facilitate the biological reaction in a bioreactor 1218 (which may be as in the embodiment of FIGS. 10 and 11) are reflected by a hot mirror 1208 and transmitted light 1206 are concentrated by a second stage reflector 120 which concentrates the light further and onto a receiver 1216.

In the preferred embodiment shown in FIG. 12 the hot mirror 1208 reflects predominantly blue light into the bioreactor while allowing predominantly red light to pass through to the second stage reflector 120 mirror collector which focuses the light onto heat exchange receiver 1216. The heat exchange receiver 1216 converts the light energy received into heat and transfers that heat to a heat exchange medium such as oil or water which may circulate 1214 through a heat exchanger 1222.

Heat from the heat exchanger may be transferred to a working fluid that energizes a turbine 1234. The solar heat source may be supplemented by natural gas 1236 as well as biomethane 1238 produced by way of fermentation 1246 of the biomass 1252 from the solar bioreactors 1218. The burning (as in a boiler 1226) of the natural gas and biomethane for the purposes of producing heat to heat a medium that drives the turbine 1234, results in emissions of carbon dioxide 1254. A selector valve 1228 may allow one or both of the fuel sources to be used. The turbine may generate electricity using a generator 1232 or do some other form of useful work such as operate an air conditioning plant. The carbon dioxide is preferably introduced into the pumping cycle of the algae culture and provide the source of non-organic carbon for the algae.

In a preferred embodiment of the system of FIG. 12, solar flux is directed to the hot mirror 1208 (or other wavelength selector) by a heliostat array (not shown). The hot mirror may be shaped in any suitable way to permit the sunlight to be directed onto the bioreactor 1218 at a suitable intensity depending on the angles of the cascade and the required optimum flux for the biological process. The final concentration received by the receiver 1216 may then be as in very high temperature systems such as 1000 C or higher. Thus, the light indicated at 1202 is already substantially concentrated.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

1. A method of generating electricity, comprising:
concentrating sunlight to a first level of solar flux;
selecting a portion of the spectrum of the light resulting therefrom and using it to photostress an organism.

2. The method of claim 1, wherein the selecting includes reflecting concentrated light from a hot mirror.

3. The method of claim 1, wherein the concentrating includes reflecting light from a heliostat array.

4. The method of claim 1, further comprising receiving the portion of the spectrum on angled surfaces forming a cascade over which liquid media, containing growth culture, trickles.

5. A method of generating electricity, comprising:
directing sunlight onto a series of angled surfaces forming a cascade;
directing liquid media carrying living organisms repeatedly over the surfaces to photo stress the organisms.

6. The method of claim 1, wherein the directing includes concentrating the sunlight to a first level of solar flux.

7. The method of claim 1, wherein the concentrating includes reflecting light from a heliostat array.

8. The method of claim 1, wherein the directing includes selecting a portion of the solar spectrum.

9. The method of claim 8, wherein the selecting includes reflecting concentrated light from a hot mirror.

10. A bioreactor, comprising:
at least one member having an array of angled surfaces forming a cascade;
the angled surfaces being irregular such that fluid flowing thereover is rendered turbulent;
a recycling channel directing fluid from the bottom of the cascade to the top such that the fluid flow down the cascade repeatedly.

11. The bioreactor of claim 10, wherein the at least one member forms gutters at intermediate points which are effective to spread fluid across the angled surfaces.

12. A method of generating electricity, comprising:
concentrating a first portion of sunlight onto a receiver which conveys a working fluid to an electrical generator;
conveying a second portion of the sunlight onto a bioreactor, the bioreactor being configured to grow an organism in the dark by selectively blocking sunlight at predefined times or in response to detected conditions and selectively unblocking the sunlight at other times or under other detected conditions.
